(19) United States
(12) Patent Application Publication
(10) Pub. No.: US 2015/0118290 A1
(43) Pub. Date: Apr. 30, 2015

(54) METHOD FOR REDUCING ESTABLISHED METASTATIC TUMOR BY PHARMACEUTICAL COMPOSITION CONTAINING POLYPEPTIDE

(71) Applicant: NATIONAL CHENG KUNG UNIVERSITY, Tainan (TW)

(72) Inventors: HUNG-CHI CHENG, Tainan (TW); CHIEN-SHENG CHEN, Taoyuan County (TW); YU-CHUAN LIN, Taichung (TW); CHIN-YUN CHENG, Miaoli County (TW); MING-MIN CHANG, Tainan (TW)

(73) Assignee: NATIONAL CHENG KUNG UNIVERSITY, Tainan (TW)

(21) Appl. No.: 14/520,381

(22) Filed: Oct. 22, 2014

Related U.S. Application Data
(63) Continuation-in-part of application No. 13/592,908, filed on Aug. 23, 2012, now abandoned.

(30) Foreign Application Priority Data
Aug. 24, 2011 (TW) 100130356
Aug. 24, 2011 (TW) 100130361
Aug. 24, 2011 (TW) 100130365

(51) Int. Cl. A61K 38/48; A61K 31/704; A61K 9/127

(52) U.S. Cl. CPC A61K 38/4813; A61K 9/127; A61K 31/704; C12Y 304/14005
USPC 424/450; 424/94.63

(57) ABSTRACT

A method for reducing an established metastatic tumor, comprising administering an effective amount of a pharmaceutical composition to a subject in need, wherein the pharmaceutical composition comprises a polypeptide of a fibronectin-binding domain of dipeptidyl peptidase IV having a maltose-binding protein (MBP) fused at an N-terminal thereof, a cross-linking molecule, an active agent, and a pharmaceutically acceptable carrier.

### CROSS REFERENCE TO RELATED APPLICATION

This application is a continuation-in-part of application Ser. No. 13/592,908, filed on Aug. 23, 2012, which claims the benefits of the Taiwan Patent Application Serial Number 100130356, 100130361 and 100130365, filed on Aug. 24, 2011, the subject matter of which is incorporated herein by reference.

### BACKGROUND OF THE INVENTION

1. Field of the Invention

The present invention relates to a method for treating cancers, more particularly, to a method for reducing an established metastatic tumor by a pharmaceutical composition containing a polypeptide.

2. Description of Related Art

Cancers are one major factor causing death around the world. However, the treatments of cancers are still very complicated and difficult to operate, since mechanisms of metastasis and growth of tumor cells among different types of cancers are different and most of them are unknown. Although there are several therapeutic methods for treating cancers, these methods still have their limits and may show side effects. For example, most of the therapeutic methods still have their limits on treating metastatic cancers.

Metastasis, also called metastatic malignant tumor, refers to the spread of tumor cells from primary organs to other non-adjacent organs via blood circulation or lymphatic system. Most studies indicate that a key mechanism of metastasis is the interaction between tumor cells and endothelial cells in secondary organs.

When tumor cells translocate from the primary organs to other organs to cause metastasis, it is hard to cure metastasis completely with surgical operation and only radiotherapy or chemotherapy can be applied to inhibit the growth of metastatic tumor cells. However, metastatic tumor cells in some important organs may show resistance to radiotherapy and continue growth, so metastasis is one major reason causing death. Till now, it is impossible to prevent cancer development completely. However, cancer mortality can be reduced significantly by (1) metastasis inhibition, and (2) effective treatment for an established metastatic tumor.

Hence, it is still desirable to provide an active agent or pharmaceutical compositions for inhibiting metastasis and tumor cell growth, in order to reduce the occurrence rate of metastasis and mortality ratio relating to cancers, even though it is hard to completely prevent the formation of cancers. In addition, it is also desirable to provide pharmaceutical compositions for diagnosing the location or degree of metastasis, in order to provide references for cancer treatment.

One of the inventors is an advisor of the graduate student whose master thesis is “Localization of fibronectin and monoclonal antibody 6a3 binding sites in the extracellular domain of rat dipeptidyl peptidase IV” (Hong, Thesis, 2007). The paper focuses on polypeptides that block the binding between DPPIV and FN for prevention or reduction of cancer metastasis. However, there is a plurality of ways to inhibit interaction between DPPIV and FN. One of the methods includes following steps. First find out binding sites between DPPIV and FN so as to learn how the two kinds of molecules are interacted with each other. Then the binding of the two molecules is blocked. For example, refer to one of the inventors' paper “A novel consensus motif in fibronectin mediates dipeptidyl peptidase IV adhesion and metastasis”, The Journal of biological chemistry, 278(27): 24600-24607, 2003, three DPPIV-binding repeats in the C-terminal of FN have been founded. The synthetic consensus DPPIV-binding sequences in the C-terminal of FN can inhibit adhesion between MTF7 tumor cells and DPPIV successfully. Furthermore, refer to another paper “Lung endothelial dipeptidyl peptidase IV promotes adhesion and metastasis of rat breast cancer cells via tumor cell surface-associated fibronectin” 273(37):24207-24215, 1998 published by The Journal of biological chemistry, it is found that the adhesion between DPPIV and FN was inhibited by a mouse monoclonal antibody (mAb) against rat DPPIV, 6A3. The 6A3 could, however, immunoprecipitate the already formed DPPIV/FN complex, suggesting that the FN- and 6A3-binding sites may differentially exist in DPPIV. Thus it is learned that there are at least two kinds of materials that block the binding between DPPIV and FN. Accordingly, the master's thesis advised by one of the inventors on 2007 (Hong, Thesis, 2007) is to elucidate the binding as well as inhibitory relationship among DPPIV, FN and 6A3. Whether FN- and 6A3-binding sites in DPPIV are the same or different from each other should be identified. In the conclusion of this paper, there are two important findings. First it is speculated that DPPIV (Asn⁴⁹-Ser⁵⁷) is the binding site for FN and DPPIV (Leu⁵⁸-Tyr⁶⁸) is a structure supporting the binding to FN. Moreover, defects occurred on DPPIV (Lys⁶⁹-Ile⁷⁴) and (Phe⁷⁷-Glu⁹⁴) will result in negative effect on the binding to FN. The second is that the 6A3-binding epitope is in rat DPPIV fragment (Asp³²⁹-Asn³³⁶) and Thr³³¹ plays a key role in its binding. Thus the paper only focuses on the finding of various polypeptides that possibly block the binding between DPPIV and FN.

In the previous research The Journal of biological chemistry, 278(27):24600-24607, 2003), the synthetic consensus DPPIV-binding sequences in the C-terminal of FN inhibit adhesion between MTF7 tumor cells and DPPIV successfully. Yet normal physical functions in the body may be affected by targeting molecules specific to DPPIV for block adhesion because DPPIV is a multi-functional protein. Moreover, it is learned that the master's thesis on 2007 has only “speculated” possible DPPIV fragments affecting the binding between DPPIV/FN and the binding between DPPIV/6A3 according to the complicated 3-dimensional structure of binding as well as inhibitory relationship among 6A3, FN, and DPP IV.

Altogether, there is a plurality of ways to inhibit interaction between DPPIV and FN, and are at least two kinds of materials that block the binding between DPPIV and FN. It is worth mentioning that although the master's thesis “Consequences after a DPP IV peptide binds to the fibronectin matrix on metastatic cancer cell surfaces” on 2009 has further showed that a recombinant peptide, DP4A, inhibited in vitro DPP IV/FN-mediated adhesion, no in vivo anti-metastatic effect was observed due to fibronectin-bound DP4A soon being endocytosed and degraded by cancer cells (Abstract and items 3-6 to 3-7 in pages 38-39). Moreover, the thesis also showed that the metastatic inhibition was apparently not due to cell growth arrest, indicating that DP4A doesn't have ability to inhibit the growth of cancer cells. Thus it is still considerable difficulty in making the DP4A as an inhibitor or a kind of treatment for reducing an already established metastatic tumor.

According to the speculation over the previous research, the inventors of the present invention has spent a lot of time (about five years) and conducted undue experiments to prepare the pharmaceutical compositions which can be used to reducing an established metastatic tumor.

Furthermore, the people skilled in the related art learn that cross-linking molecules and carriers on medical uses are selected according to the materials they carried. In fact, there are hundreds of combinations of cross-linking molecules with carriers. The cross-linking molecule included in the pharmaceutical compositions of present invention is SM(PEG)₂₄ and the acceptable carrier is liposome, preferably ATA-tagged liposome. The pharmaceutical compositions mentioned above is the most optimal combination found and prepared by the inventors of the present invention after taking a lot of time and undue experiments. The inhibition effect of the pharmaceutical compositions on the established metastatic tumor also has been confirmed herein.

### SUMMARY OF THE INVENTION

An object of the present invention is to a method for reducing an established metastatic tumor, comprising administering an effective amount of a pharmaceutical composition to a subject in need, wherein the pharmaceutical composition comprises a polypeptide of a fibronectin-binding domain of dipeptidyl peptidase IV having a maltose-binding protein (MBP) fused at an N-terminal thereof, a cross-linking molecule, an active agent, and a pharmaceutically acceptable carrier, and wherein the cross-linking molecule is SM(PEG).sub.24, and the pharmaceutically acceptable carrier is liposome.

According to an embodiment of the present invention, the fibronectin-binding domain of dipeptidyl peptidase IV has a sequence with 70-100% similarity to a sequence represented by SEQ ID NO: 1.

According to an embodiment of the present invention, the fibronectin-binding domain of dipeptidyl peptidase IV has a sequence with 70-100% identity to a sequence represented by SEQ ID NO: 1.

According to an embodiment of the present invention, the fibronectin-binding domain of dipeptidyl peptidase IV has a sequence represented by SEQ ID NO: 1.

According to an embodiment of the present invention, the fibronectin-binding domain of dipeptidyl peptidase IV is a binding domain A of dipeptidyl peptidase IV.

According to an embodiment of the present invention, the active agent is doxorubicin.

According to an embodiment of the present invention, the liposome is ATA-tagged liposome.

Another object of the present invention is to provide a polypeptide harboring FN-binding domain for inhibiting, treating or diagnosing metastasis, which can specifically bind to pericelluar fibronectin of metastatic cancer cells, and therefore the polypeptide can inhibit the interaction between cancer pericellular fibronectin (FN) and endothelial cells of secondary organs.

Another object of the present invention is to provide an use of a polypeptide to prepare a pharmaceutical composition for inhibiting, treating or diagnosing metastasis, which can inhibit a metastatic rate of tumor cells or be used to detect whether metastasis happens or not.

Another object of the present invention is to provide a pharmaceutical composition for inhibiting metastasis. The polypeptide contained therein can specifically bind to metastatic tumor cells expressing pericellular fibronectin to inhibit the metastasis or tumor cells.

Another object of the present invention is to provide a pharmaceutical carrier for recognizing tumor cells expressing pericellular fibrinectin. The polypeptide contained in the pharmaceutical carrier can bind specifically to cancer pericellular fibronectin. Hence, when the polypeptide is used with conventional drug carrier to form the pharmaceutical carrier of the present invention, the fibronectin on metastatic tumor cells can be specifically recognized by the polypeptide.

Another object of the present invention is to provide a pharmaceutical composition for treating or preventing cancers, which comprises the aforesaid pharmaceutical carrier for recognizing tumor cells expressing fibronectin and an active agent for treating cancers. The polypeptide of the pharmaceutical carrier can specifically recognize fibronectin on metastasis tumor cells. When the polypeptide of the pharmaceutical carrier binds to fibronectin, the whole pharmaceutical composition can enter into tumor cells via endocytosis thereof. Hence, an effect of locally and specifically treating cancers can be accomplished.

Another object of the present invention is to provide a pharmaceutical composition for diagnosing metastasis, which comprises the aforesaid pharmaceutical carrier for recognizing tumor cells expressing fibronectin and a marker. When the polypeptide of the pharmaceutical carrier binds to fibronectin, the whole pharmaceutical composition can enter into tumor cells via endocytosis thereof. Hence, it is possible to detect the metastatic degree and the localization of tumor cells via the marker of the pharmaceutical composition.

To achieve the aforesaid objects, the present invention provides a polypeptide for inhibiting, treating or diagnosing metastasis, which is a polypeptide, a derivative polypeptide, or a mutated polypeptide of a fibronectin-binding domain of dipeptidyl peptidase IV.

In addition, the present invention also provides a use of the aforesaid polypeptide, which is used to prepare a pharmaceutical composition for inhibiting, treating or diagnosing metastasis.

The present invention further provides a pharmaceutical composition for inhibiting, treating or diagnosing metastasis, which comprises: an effective amount of the aforesaid polypeptide, and a pharmaceutically acceptable carrier.

In the aforesaid pharmaceutical composition of the present invention, the pharmaceutical acceptable carrier can be at least one selected from the group consisting of activators, excipients, adjuvants, dispersants, wetting agents, suspensions, liposome, micelle, microsphere, nanoparticle, and dendrimer. When the pharmaceutically acceptable carrier is activators, excipients, adjuvants, dispersants, wetting agents, suspensions, or a combination thereof, it can facilitate or stabilize the activity of polypeptide of the present invention. When the pharmaceutical acceptable carrier is liposome, micelle, microsphere, nanoparticle, dendrimer, or a combination thereof, the pharmaceutically acceptable carrier can be used to carry an active agent or a marker to accomplish the purpose of inhibiting, treating or diagnosing metastasis. In one aspect of the present invention, when the pharmaceutical composition of the present invention further comprises an active agent such as anti-cancer drugs or a marker, the active agent or the marker may be encapsulated in the liposome or the like. Therefore, the active agent or the marker encapsulated in the liposome may release therefrom to obtain the purpose of inhibiting, treating or diagnosing metastasis as the pharmaceutical composition is administered into a subject in need. In another aspect of the present invention, when the pharmaceutical composition of the present invention further comprises an active agent or a marker, the active agent or the marker as well as the polypeptide of the present invention may directly link to the pharmaceutical carrier such as micelle, microsphere, nanoparticle, or dendrimer. Hence, the active agent or the marker linked to the pharmaceutical composition may release therefrom to obtain the purpose of the inhibiting, treating or diagnosing metastasis as the pharmaceutical composition is administered into a subject in need. However, the linkage between the pharmaceutical carrier, the active agent or the marker, and the polypeptide of the present invention may be different according to the therapeutic requirement, and the present invention is not limited thereto.

Furthermore, the present invention provides a pharmaceutical carrier for recognizing tumor cells expressing fibronectin, which comprises: a drug carrier and the aforesaid polypeptide linked to the surface of the drug carrier.

The present invention also provides a pharmaceutical composition for preventing or treating metastasis, which comprises: the aforesaid pharmaceutical carrier for recognizing tumor cells, and an active agent contained in the pharmaceutical carrier, wherein the active agent is used to treat cancer, and the tumor cells can express fibronectin on the surface thereof.

In addition, the present invention further provides a pharmaceutical composition for diagnosing metastasis, which comprises: the aforesaid pharmaceutical carrier for recognizing tumor cells, and a marker contained in the pharmaceutical carrier, wherein the marker is used to identify the localization of tumor cells, and the tumor cells can express fibronectin on the surface thereof.

Furthermore, the present invention also provides a method for treating, preventing or diagnosing metastasis, which comprises: administering an effective amount of the aforesaid pharmaceutical composition to a subject in need. Preferably, the subject is human.

It is found that metastatic tumor cells bind to endothelial cells of secondary organs via interactions between fibronectin on metastatic tumor cells and endothelial cells, and spread or metastasize to secondary organs. The polypeptide of the present invention, which is derived from a fibronectin-binding domain of dipeptidyl peptidase IV (DPPIV) can bind specifically to fibronectin on metastatic tumor cells. The binding of the fibronectin-binding domain of DPPIV polypeptide to fibronectin can inhibit the interactions between the metastatic tumor cells and endothelial cells of secondary organs, so the metastatic rate of tumor cells can further be decreased.

In the present invention, the fibronectin-binding domain of DPPIV can be a domain binding specifically to fibronectin on metastatic tumor cells. Preferably, the fibronectin-binding domain of dipeptidyl peptidase IV is a binding domain A of dipeptidyl peptidase IV (DPPIV A domain, DP4A).

The inventors of the present invention have made several polypeptide fragments of DPPIV with different sequence lengths, and found the binding domain A of DPPIV therefrom. The sequence of the binding domain A of DPPIV is represented by SEQ ID NO: 1. In addition, the sequence of DPPIV can also be accessed from the website of National Center for Biotechnology Information (NCBI), and the accessing number thereof is NP_{—}036921.1. The binding domain A of DPPIV is a fragment from 29 to 130 amino acid residues of DPPIV. Since the sequences of DPPIVs among different species may be slightly different, a person skilled in the art can tell the similarities between DPPIVs of different species and the sequence represented by SEQ ID NO. 1 of the present invention via sequence alignment software such as ClustalW or NCBI BLAST. Besides, when the interaction between the binding domain A of DPPIV and fibronectin is predicted through other bioinformatics software, a person skilled in the art can understand that either the polypeptide of the present invention or other polypeptides similar thereto can specifically bind to fibronectin. More specifically, any mutations of the amino acid residues with similar properties in the binding domain A of DPPIV (for example, arginine substituted with asparagine), which do not influence the interaction between the binding domain A and fibronectin, belong to the scope of the present invention. Hence, any proteins, protein fragments or polypeptides having sequences with 70% or more sequence similarity to the sequence represented by SEQ IN NO. 1 can obtain the aforesaid objects of the present invention. Preferably, the fibronectin-binding domain (i.e. the binding domain A) of dipeptidyl peptidase IV has a sequence with 70-100% identity to a sequence represented by SEQ ID NO: 1.

In the present invention, the term “similarity” refers to the percentage of similar amino acid residues. Not only identical amino acid residues, but also the amino acid residues with similar properties are defined as similar amino acid residues. Additionally, the term “identity” refers to the percentage of identical amino acid residues.

In addition, for the gene system selection and the convenience of protein expression, the binding domain A of DPPIV can be expressed with other conventional expressing proteins. For example, when an E. coli expression system is used, the domain A of DPPIV (DP4A) can be expressed together with a maltose-binding protein (MBP) to form a fused protein of DP4A-MBP.

Hence, in the pharmaceutical compositions for recognizing tumor cells expressing fibronectin or for preventing, treating or diagnosing metastasis of the present invention, the polypeptide may further comprise a maltose-binding protein.

In addition, in the pharmaceutical carrier for recognizing tumor cells expressing fibronectin, the polypeptide can link to the drug carrier via a cross-linking molecule. In one aspect of the present invention, the cross-linking molecule links to the drug carrier and the fused protein of DP4A-MBP via covalent bonds. Herein, the cross-linking molecule can be SM(PEG)ₙ (NHS-(PEG)ₙ-maleimide), CA(PEG)ₙ (car-boxyl-(PEG)ₙ-amine) or other similar cross-linking molecules, and n is an integral of 2-24. However, the present invention is not limited thereto.

Furthermore, the drug carrier of the pharmaceutical carrier of the present invention can be liposome, micelle, microsphere, nanoparticle, dendrimer or a combination thereof.

The active agent used in the pharmaceutical composition for preventing or treating metastasis can be any cytotoxic agents, or the pharmaceutical composition can be used with radiotherapy or immunotherapy. Examples of cytotoxic agents include: topoisomerase inhibitor such as etoposide, teniposide, camptothecin, topotecan, irinotecan, doxorubicin and daunorubicin; antimetabolites such as capecitibine, gemcitabine, 5-fluorouracil or 5-fluorouracil/leucovorin, fludarabine, cytarabine, mercaptopurine, thioguanine, pentostatin and methotrexate; taxanes such as paclitaxel and docetaxel; vinca alkaloid such as vincristine and vinblastine; platinum agents such as cisplatin, carboplatin and oxaliplatin; alkylating agents such as melphalan, chlorambucil, busulfan, thiotepa, ifosfamide, carmustine, lomustine, semustine, streptozocin, decarbazine and cyclophosphamide; antibiotics such as actinomycin D, bleomycin, mitomycin C, adriamycin, daunorubicin, idarubicin, doxorubicin and pegylated liposome doxorubicin; thalidomide and the like such as CC-5013 and CC-4047; antibodies such as trastuzumab, rituximab, cetuximab and bevacizumab; tyrosine protein kinase inhibitors such as imatinib mesylate, gefitinib, dasatinib, erlotinib, lapatinib, sunitinib, nilotinib and sorafenib; mitoxantrone; dexamethasone; prednisone; and temozolomide.

In addition, the marker used in the pharmaceutical composition for diagnosing metastasis of the present invention can be any contrast medium generally used in the art, for example: X-ray contrast agents such as barium-based agents, ionic iodine-based agents and nonionic iodine-based agents; magnetic resonance imaging (MRI) contrast agents such as gadolinium agents; and contrast-enhanced ultrasound agents such as octafluoropropane.

Furthermore, in the pharmaceutical compositions of the present invention, the polypeptide or the pharmaceutical carriers may be used with any pharmaceutically acceptable carriers, such as activators, excipients, adjuvants, dispersants, wetting agents, and suspensions.

In the pharmaceutical compositions of the present invention, the term “pharmaceutically acceptable carrier” means that the carrier must be compatible with the active ingredients (and preferably, capable of stabilizing the active ingredients) and not be deleterious to the subject to be treated. The carrier may be at least one selected from the group consisting of active agent, adjuvants, dispersants, wetting agents and suspending agents. The example of the carrier may be microcrystalline cellulose, mannitol, glucose, non-fat milk powder, polyethylene, polyvinylpyrrolidone, starch or a combination thereof.

In addition, the term “treating” used in the present invention refers to the application or administration of the pharmaceutical compositions containing the polypeptide or the active agents to a subject with symptoms or tendencies of suffering from cancer in order to cure, heal, alleviate, relieve, alter, remedy, ameliorate, improve, prevent or affect the symptoms or tendencies of cancers. Furthermore, “an effective amount” used herein refers to the amount of each active ingredients such as polypeptide or the active agents contained in the pharmaceutical carrier required to confer therapeutic effect on the subject. The effective amount may vary according to the route of administration, excipient usage, and co-usage with other active ingredients.

The pharmaceutical compositions and the method of the present invention can be used to treat cancers including solid cancers or hematologic malignancies. Examples of solid cancers include: prostate cancer including androgen-dependence and androgen-independence prostate cancer; pancreas cancer; bladder cancer; colon cancer; breast cancer including metastatic breast cancer; kidney cancer including metastatic kidney cancer; liver cancer; lung cancer including non-small cell lung cancer (NSCLC), bronchioloalveolar carcinoma (BAC), squamous cell lung cancer and adenocarcinoma; stomach cancer; esophageal cancer; head and neck cancer including head and neck squamous cell carcinoma; ovarian cancer including epithelial ovarian cancer and primary peritoneal carcinoma; cervical cancer; melanoma; neuroendocrine cancer including metastatic neuroendocrine cancer; bone cancer; and soft tissue sarcoma. Examples of hematologic malignancies include: acute myeloid leukemia (AML); chronic myeloid leukemia (CML) including accelerated phase CML, and CML in blast phase (CML-BP); brain cancer including ganglioglioma, oligodendroglioma, glioblastoma multiform and astrocytoma; acute lymphocytic leukemia (ALL); chronic lymphocytic leukemia (CLL); Hodgkin's disease (HD); multiple myeloma (MM); Waldenstorm's macroglobulinemia; non-Hodgkin's lymphoma (NHL) including follicular lymphoma and mantic cell lymphoma; B lymphoblastic lymphoma; T lymphoblastic lymphoma; myelodysplastic syndromes (MDS) including refractory anemia (RA), refractory anemia with ringed siderblasts (RARS), refractory anemia with excess blasts (RAEB) and refractory anemia with excess blasts in transformation (RAEB-T); and myelodysplastic syndrome.

In addition, the pharmaceutical compositions of the present invention can be administered via parenteral, inhalation, local, rectal, nasal, sublingual, or vaginal delivery, or implanted reservoir. Herein, the term “parenteral delivery” includes subcutaneous, intradermic, intravenous, intra-articular, intra-arterial, synovial, intrapleural, intrathecal, local, and intracranial injections.

Other objects, advantages, and novel features of the invention will become more apparent from the following detailed description when taken in conjunction with the accompanying drawings.

### BRIEF DESCRIPTION OF THE DRAWINGS

This patent application contains color drawings executed in color. Copies of this patent application publication with color drawings will be provided by the Office upon request and payment of the necessary fee.

FIG. 1A is a result of western blot showing the interaction between DP4A and fibronectin gelatin-beads or control gelatin-beads according to Example 1 of the present inventions;

FIG. 1B is a result of western blot showing the interaction between DP4A with different concentrations and fibronectin gelatin-beads according to Example 1 of the present invention.

FIG. 1C is a result showing the binding strength curve of DP4A to fibronectin according to Example 1 of the present invention;

FIG. 2A is a fluorescence staining and colocalization result showing the interaction between DP4A and fibronectin expressed by adherent MTF7 cells according to Example 2 of the present invention;

FIG. 2B is a fluorescence staining and colocalization result showing the interaction between DP4A and fibronectin expressed by adgerent 4T1 cells according to Example 2 of the present invention.

FIG. 3A is a fluorescence staining and colocalization result showing the interaction between DP4A and fibronectin expressed by suspended MTF7 cells according to Example 2 of the present invention;

FIG. 3B is a fluorescence stainging and coloralization result showing the interaction between DP4A and fibronectin expressed by suspended 4T1 cells according to Example 2 of the present invention;

FIG. 4A is a fluorescence staining showing binding of DP4A to LL2 and 4T1 cells according to Example 3 of the present invention;

FIG. 4B is a result of cell adhesion assay of 4T1 cells to DPPIV in the presence of DP4A or the control protein MBP according to Example 3 of the present invention;

FIG. 4C is a result of cell adhesion assay of LL2 cells to DPPIV in the presence of DP4A or the control protein MBP according to Example 3 of the present invention;

FIG. 5 is a result of in vivo experimental metastasis assay wherein LL2 cells and DP4A or the control protein MBP were concomitantly injected through tail vein according to Example 5 of the present invention;

FIGS. 6(a)-6(d) show fluorescent photos of LL2 cells according to Example 6 of the present invention, wherein FIG. 6(a) is a fluorescent photo showing LL2 tumor cells treated with MBP-liposome, FIG. 6(b) is a fluorescent plus phase contrast photo showing LL2 tumor cells treated with MBP-liposome, FIG. 6(c) is a fluorescent photo showing LL2 tumor cells treated with DP4A-liposome, and FIG. 6(d) is a fluorescent plus phase control photo showing LL2 tumor cells treated with DP4A-liposome;

FIGS. 7(a)-7(d) show fluorescent photos of tissue pieces according to Example 7 of the present invention, wherein FIG. 7(a) is a fluorescent photo in which the DP4A-liposome was intravenously injected after the metastasis of LL2 cells in the mouse lungs was occurred, FIG. 7(b) is a phase contrast photo in which DP4A-liposome was intravenously injected after the metastasis of LL2 cells in the mouse lungs was occurred, FIG. 7(c) is a fluorescent photo that MBP-liposome was intravenously injected after the metastasis of LL2 cells in the mouse lungs was occurred, and FIG. 7(d) is a phase contrast photo in which MBP-liposome was intravenously injected after the metastasis of LL2 cells in the mouse lungs was occurred;

FIGS. 8(a)-8(d) show fluorescent photos of tissue pieces in which LL2 tumor cells were transfected with GFP that were intravenously injected according to Example 7 of the present invention, wherein FIG. 8(a) is a fluorescent photo in which LL2 tumor cells were indicated by GFP, FIG. 8(b) is a fluorescence photo in which DP4A-liposome were intracellularly delivered in the same LL2 cells as in FIG. 8(a) after the metastasis of LL2 cells in the mouse lungs was occurred, FIG. 8(c) is a merged photo of tumor cells and DP4A-liposomes, and FIG. 8(d) is a phase contrast photo of lung pieces;

FIGS. 9(a)-9(d) show fluorescent photos of LL2 cells according to Example 9 of the present invention, wherein FIG. 9(a) is a fluorescent photo showing LL2 tumor cells treated with DP4A-lipo-dox, FIG. 9(b) is a fluorescent plus phase contrast photo showing LL2 tumor cells treated with DP4A-lipo-dox, FIG. 9(c) is a fluorescent photo showing LL2 tumor cells treated with MBP-lipo-dox, and FIG. 9(d) is a fluorescent plus phase contrast photo showing LL2 tumor cells treated with DP4A-lipo-dox;

FIG. 10 shows a photo of MTF7 tumor cells treated with MBP-lipo-dox and DP4A-lipo-dox respectively;

FIGS. 11(a)-11(d) show phase contrast photos of LL2 and 4T1 tumor cell treated with MBP-lipo-dox or DP4A-lipo-dox, wherein FIG. 11(a) is a phase contrast photo of LL2 treated with MBP-lipo-dox, FIG. 11(b) is a phase contrast photo of LL2 treated with DP4A-lipo-dox, FIG. 11(c) is a phase contrast photo of 4T1 cells with MBP-lipo-dox, and FIG. 11(d) is a phase contrast photo of 4T1 cells with DP4A-lipo-dox;

FIG. 12 shows a photo of lungs taken from mice according to Example 10 of the present invention, wherein the upper panel represents lungs taken from mice treated with MBP-lipo-dox, and the lower panel represents those treated with DP4A-lipo-dox;

FIG. 13A is a quantitative result showing the mouse lung weights according to Example 10 of the present invention; and

FIG. 13B is a quantitative result showing the tumor nodule numbers in the lungs taken from mice with cancer metastases according to Example 10 of the present invention.

### DETAILED DESCRIPTION OF THE PREFERRED EMBODIMENT

An object of the present invention is to a method for reducing an established metastatic tumor, comprising administering an effective amount of a pharmaceutical composition to a subject in need, wherein the pharmaceutical composition comprises a polypeptide of a fibronectin-binding domain of dipeptidyl peptidase IV having a maltose-binding protein (MBP) fused at an N-terminal thereof, a cross-linking molecule, an active agent, and a pharmaceutically acceptable carrier, and wherein the cross-linking molecule is SM(PEG).sub.24, and the pharmaceutically acceptable carrier is liposome.

According to an embodiment of the present invention, the fibronectin-binding domain of dipeptidyl peptidase IV has a sequence with 70-100% similarity to a sequence represented by SEQ ID NO: 1.

According to an embodiment of the present invention, the fibronectin-binding domain of dipeptidyl peptidase IV has a sequence with 70-100% identity to a sequence represented by SEQ ID NO: 1.

According to an embodiment of the present invention, the fibronectin-binding domain of dipeptidyl peptidase IV has a sequence represented by SEQ ID NO: 1.

According to an embodiment of the present invention, the fibronectin-binding domain of dipeptidyl peptidase IV is a binding domain A of dipeptidyl peptidase IV.

According to an embodiment of the present invention, the active agent is doxorubicin.

According to an embodiment of the present invention, the liposome is ATA-tagged liposome.

The present invention has been described in an illustrative manner, and it is to be understood that the terminology used in intended to be in the nature of description rather than of limitation. Many modifications and variations of the present invention are possible in light of the above teachings. Therefore, it is to be understood that within the scope of the appended claims, the invention may be practiced otherwise than as specifically described.

### Preparative Embodiment 1

### Construct, Expression and Purification of MBP and MBP-DP4A

Gene Sequence of dipeptidyl peptidase IV (DPPIV) can be found on the website of National Center of Biotechnology Information. The DPPIV used in the present embodiment was derived from Rat, wherein the Gene_ID is 25253, mRNA Accession No. is NM_{—}012789.1, and protein Accession No. is NP_{—}036921.1.

The amino acid sequence of the binding domain A of DPPIV (DP4A) is from 29 to 130 amino acid residues of DPPIV represented by SEQ IN NO:1, and the corresponding open reading frame thereof is a NDA sequence from 85 to 390 nucleotides of DPPIV represented by SEQ IN NO:2.

SEQ IN NO: 1:
NKDEAAADSRRTYTLADYLKNTFRVKSYSLRWVSDSEYLYKQENN
ILLFNAEHGNSSIFLENSTFEIFGDSISDYSVSPDRLFVLLEYNY
VKQWRHSYTASY
SEQ IN NO: 2:
aacaaagatgaagcggccgctgatagccgcagaacttacacacta
gctgactatttaaagaatacctttcgggtcaagtcctactccttg
cggtgggtttcagattctgaatacctctacaagcaagaaaacaat
atcttgctattcaatgctgaacacgggaacagctccattttcttg
gagaacagacctttgagatctttggagattctataagtgattatt
cagtgtcaccggacagactgttcgttctcttagaatacaattatg
tgaagcaatggagacactcctacacggcttcatac

A single colony of E. coli contacting DP4A gene recombination was seeded in LB/A medium (3 mL) and suspension cultured in an incubator at 37° C. For 12-16 hr (200 rpm). The cultured medium was separated at 13000 rpm for 1 min, and then the supernatant was removed. Next, a small amount of plasmid NDA was obtained by using a High-Speed Plasmid Mini Kit (Geneaid) according to the protocol thereof.

Forward and reverse primers for the DP4A gene recombination, and the obtained plasmid DNA as a template were used to perform a polymerase chain reaction (PCR), and Taq polymerase was used to amplify the nucleotide fragment of the DP4A gene recombination. Next, the amplified fragment was checked and recycled with agarose gel electrophoresis and gel/PCR DNA fragments extraction kit.

An E. coli expression vector, pMAL-c2× with restriction cutting sites of EcoRI and SacII was used. Next, restriction enzymes of EcoRI and SacII were performed on the nucleotide fragment of the DP4A gene recombination and pMAL-c2×, and then a construct inserted with DP4A gene recombination was obtained by using 4 ligase. The obtained construct was transformed into competent cells, and then the obtained competent cells were cultured in LB/A medium. The obtained colonies were checked with colony PCR to ensure whether the transformation was successful or not. The construct in the bacteria solution with successful transformed competent cells was separated with High-Speed Plasmid Mini Kit, and the sequence accuracy of the construct was checked with DNA sequencing.

The construct with accurate sequence containing the DP4A gene recombination and pMAL-c2× expressing maltose-binding protein (MBP) were transformed respectively into E. coli BL 21 competent cells. The two cells were cultured and amplified, and then Isopropyl B-D-1-thiogalactopyranoside (IPTG) was added into the culture medium to induce the protein expression. Next, the bacteria solutions were lysed, and the obtained lysate was injected into a column containing amylose resin. The column was washed with a column buffer, and a maltose solution (10 mM) was used to elute the recombinant proteins.

Since the size of the DP4A polypeptide is small, the DP4A gene recombination was inserted into pMAL-c2× expressing maltose binding protein (MBP) to obtain a recombinant protein, which is a fused protein of MBP and DP4A(MBP-DP4A). The amino acid sequence of MBP-DP4A is represented by SEQ IN NO: 3.

SEQ IN NO: 3
MKIEEGKLVIWINGDKGYNGLAVEGKKFEKDTGIKVTVEHPDKLE
EKFPQVAATGDGPDIIFQAHDRFGGYAQSGLLAEITPDKAFQDKLY
PFTWDAVRYNGKLIAYPIAVEALSLIYNKDLLPNPPKTWEEIPALD
KELKAKGKSALMFNLQEPYFTWPLIAADGGYAFKYENGKYDIKD
VGVDNAGAKAGLTFLVDLIKNKHMNADTDYSIAEAAFNKGETAM
TINGPWAWSNIDTSKVNYGVTVLPTFKGQPSKPFVGVLSAGINAA
SPNKELAKEFLENYLLTDEGLEAVNKDKPLGAVALKSYEEELAKD
PRIAATMENAQKGEIMPNIPQMSAFWYAVRTAVINAASGRQTVDE
ALKDAQTNSSSNNNNLGIEGRISEF

[symbol]

[symbol]

[symbol]

[symbol]

(The amino acid residues shown in italic and
bold are the polypeptide of DP4A)

### Example 1

### In Vitro Binding Assay of DP4A and Fibronectin on Metastatic Tumor Cells

Gelatin-beads linked with fibronectin to mimic pericellular fibronectin of metastatic tumor cells with similar biochemical characteristics were obtained, and the preparation process is shown as follows. First, 30 μl of gelatin sepharose was washed with PBS/0.05% Triton X-100 for three times. Next, fibronectin was added and mixed with washed gelatin sepharose at 4° C. For 3 days to obtain fibronectin gelatin-beads.

Then, the fibronectin gelatin-beads and control gelatin-beads without linked with fibronectin were respectively washed with 50 ml of PBS/0.05% Triton X-100. 7 μl of fibronectin gelatin-beads and control gelatin-beads were respectively mixed with MBP-DP4A dissolved in 300 μl of PBS/0.05% Triton X-100, and reacted at 4° C. overnight. The samples were separated and washed with PBS/0.05% Triton X-100 for several times, and the obtained precipitants were analyzed with protein electrophoresis and western blot. For the western blot, mouse anti-MBP antibody was used as a primary antibody.

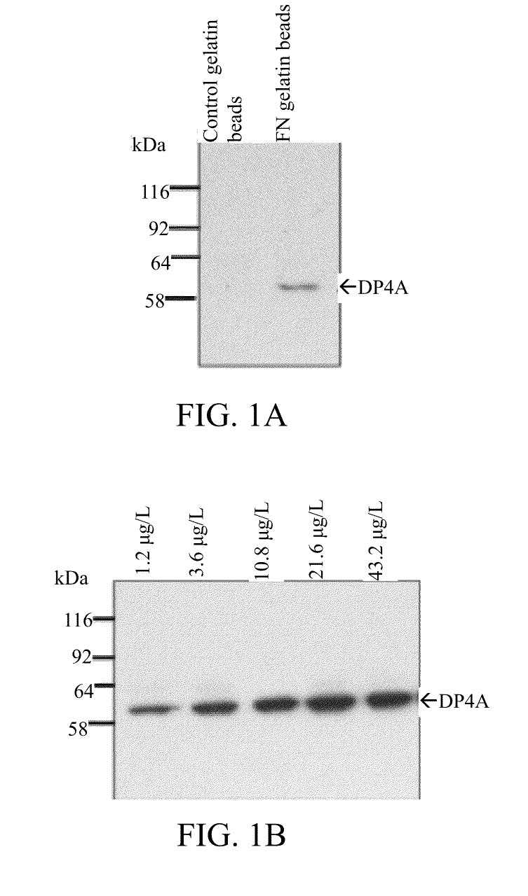

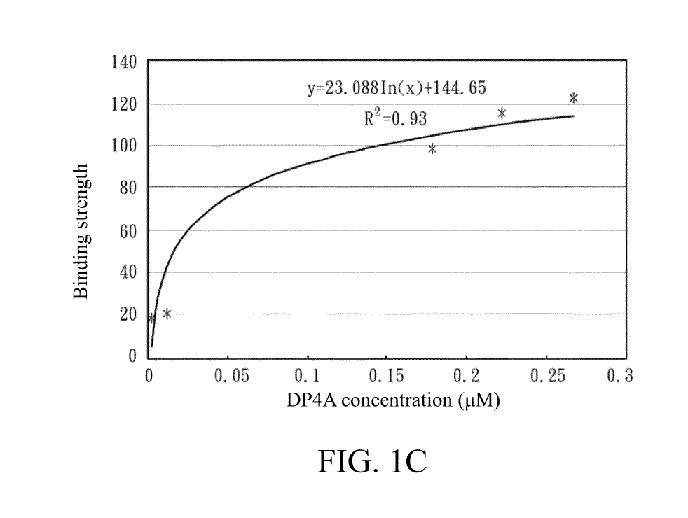

As shown in FIG. 1A, DP4A can be pulled down by fibronectin gelatin-beads, but cannot be pulled down by control gelatin-beads. This result indicates that DP4A can interact with fibronectin on metastatic tumor cells.

In addition, 1.2, 3.6, 10.8, 21.6 and 43.2 μg/L of DP4A was reacted with fibronectin gelatin-beads, and the experimental results were examined with western blot, as shown in FIG. 1B. The DP4A detected by western blot was quantified with Image J to obtain a parabolic tropic curve, as shown in FIG. 1C. The average K_{d} is about 0.0267 μM based on three repeated experiments. These results show that DP4A can bind specifically to fibronectin on metastatic tumor cells.

### Example 2

In Vitro Binding Assay of DP4A and Adherent Metastatic Tumor Cells Expressing Fibronectin

First, MBP-DP4A and MBP (control) was reacted with sufficient biotin at 4° C. for 2 hr, and the obtained samples were dialyzed with PBS buffer to obtain biotin-labeled MBP-DP4A and MBP. Hereafter, the biotin-labeled MBP-DP4A and MBP were respectively shown as Bio-DP4A and Bio-MBP.

A vector with FN-GFP gene and capable of expressing in mammalian cells, pAIPEN (1 μg) was transfected into rat breast cancer cells MTF7 or mouse mammary tumor cells 4T1 by electroporation. The transfected cells were seeded in plates, and FN-GFP protein secreted from tumor cells after 2-3 days.

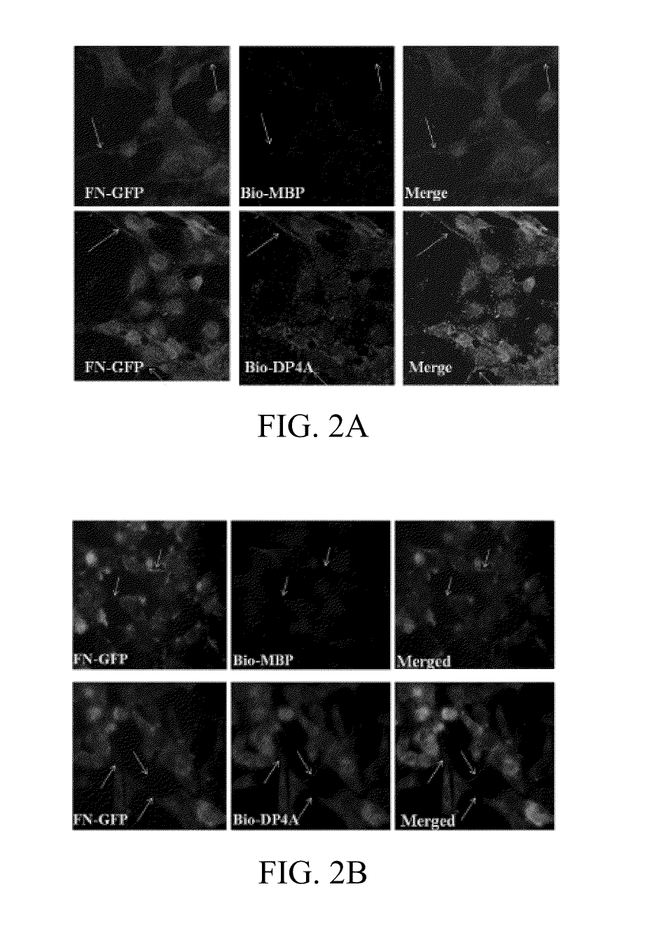

The tumor cells expressing FN-GFP protein were washed PBS buffer, fixed with 4% paraformaldehyde at room temperature for 10 min, and washed with PBS buffer. Then, Bio-DP4A and Bio-MBP were added into the solution containing the tumor cells expressing FN-GFP protein, and the cells were cultured in an incubator at 37° C. for 1 hr. After the culture medium was washed and the cells were fixed with 4% paraformaldehyde, the fixed cells were visualized with fluorescence staining. The results are shown in FIG. 2A and FIG. 2B.

### Example 3

### In Vitro Binding Assay of DP4A and Suspended Metastatic Tumor Cells Expressing Fibronectin

A vector with FN-GFP gene and capable of expressing in mammalian cells, pAIPEN (1 μg) was transfected into MTF7 or 4T1 tumor cells by electroporation. The transfected cells were seeded in plates, and FN-GFP protein secreted from tumor cells after 2-3 days.

MTF7 tumor cells expressing FN-GFP protein were scraped from plates to acquire the single cell suspension, or 4T1 tumor cells expressing FN-GFP protein were trypsinized and recovered in the secreted FN-GFP-containing growth media in single cell suspension at 37° C. For 2 hrs. The obtained tumor cells washed with PBS buffer, and Bio-DP4A and Bio-MBP were added into the solution containing the tumor cells. The obtained samples were placed in a PBS buffer containing 1% BSA and cultured at 4° C. For 1 hr.

The cells were separated with centrifuge and washed with cold PBS buffer, and then the samples were placed in a PBS buffer containing 1% BSA and reacted with Streptavidin-PE at 4° C. for 1 hr.

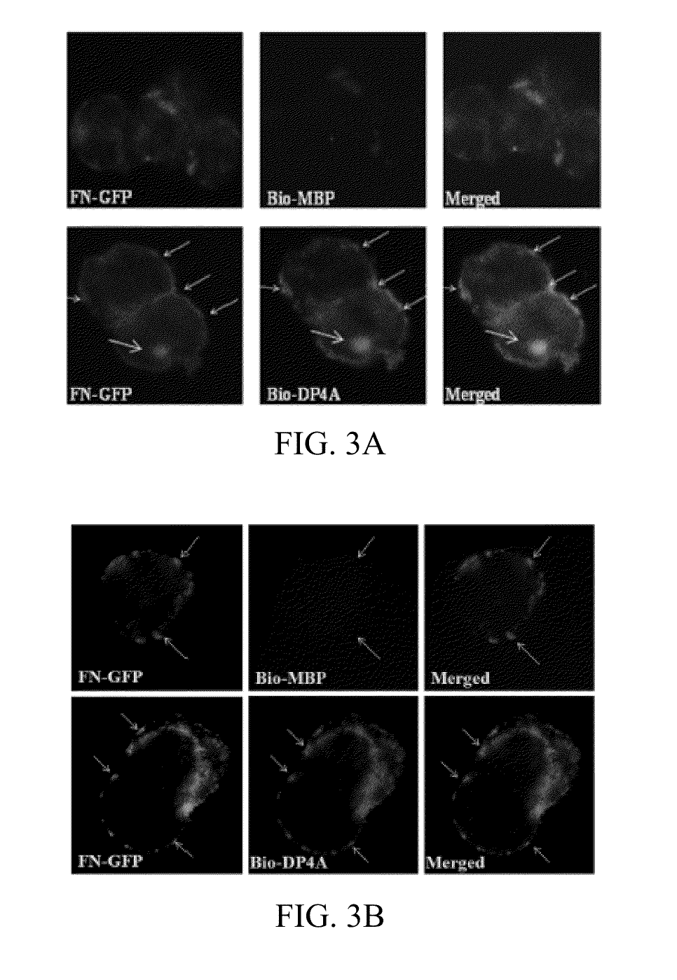

The obtained cells were separated with centrifuge and fixed with 2% paraformaldehyde on ice. The fixed cells were visualized with fluorescence staining. The results are shown in FIG. 3A and FIG. 3B.

As shown in FIGS. 2A, 2B, 3A and 3B, MTF7 and 4T1 tumor cells can express FN-GFP protein (green fluorescence), and a large amount of Bio-DP4A protein can be observed on cells (red fluorescence). When the cells were observed with confocal microscope, a colocalization of FN and Bio-DP4A can be found (as shown in yellow arrows) as FN-GFP protein was interacted with Bio-DP4A or Bio-MBP.

The results of Examples 2 and 3 show that the assembled FN on adherent and suspended tumor cells can specifically colocalize with DP4A when observed with fluorescence staining

### Example 4

### Cell Adhesion Assay for DP4A Influencing the Interaction Between Tumor Cells and Full-Length DFPIV

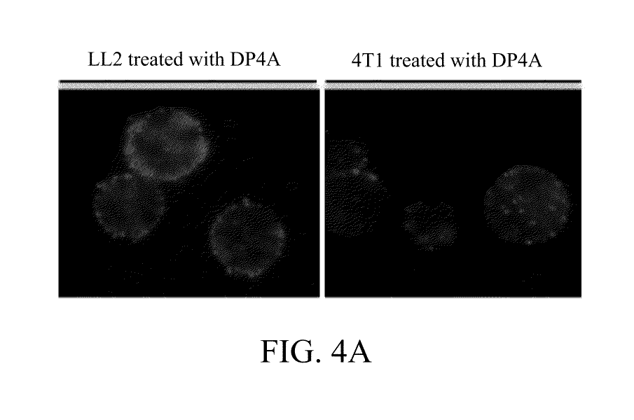

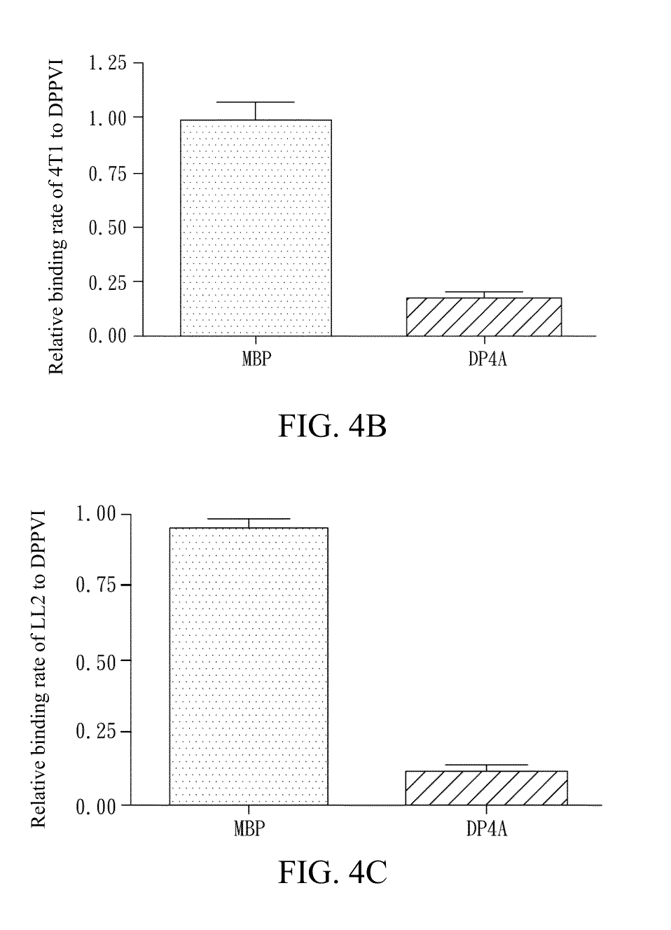

In the present example, metastatic lung tumor cells LL2 or 4T1 were used for the present cell adhesion assay. Suspended cultured LL2 or 4T1 tumor cells were mixed with Bio-DP4A, and the cells were visualized with fluorescence staining. As shown in FIG. 4A, LL2 and 4T1 tumor cells can interact with DP4A.

In addition, full-length DDPIV derived from rat kidney or lungs was used in the present example. The extraction process for full-length DDPIV was shown as follows.

Rat kidney or lung was mixed with lysis buffer containing 2% NP40 (25 ml), PMSF (1M, 50 μl) and 2% Leupeptin (500 μl) was added into the solution, and the organs were smashed with a homogenizer. After performing washing and separating processes, the obtained supernatant was filtered. The filtered sample was purified through column chromatography with protein G microbeads and anti-DPP IV mAmb 6A3 microbeads for several times to obtain purified DPPIV.

ELISA plates were coated with 30 μl of 1 mg/ml BSA. 0.2 mg/ml DPPIV, or 0.5 mg/ml poly-L-Lysine, and incubated at 4° C. Over night. Then, PBS buffer containing 1% BSA was added into plates, and then the trypsinized cells from the plates were recovered in 20% FBS DMEM at 37° C. For 2 hrs and washed with adequate PBS buffer.

Next, LL2 and 4T1 tumor cells treated with MBP-DP4A and MBP (7.5 μg/ml) were suspended in 100 μl of DMEM, seeded in ELISA plates (4.5×10⁴ cells/well), and cultured in an incubator containing 5% CO₂ at 37° C. After 0.5-1 hr, the adhesion of tumor cells was observed.

After suspended cells were removed and ELISA plates were washed with PBS buffer, the cells in each well were fixed with 50 μl of crystal violet for 10 min. After washing with PBS, the absorptions corresponding to 492 nm were determined. The background-subtracted absorptions from DPP IV-coated wells harboring the bound cells were divided by the background-subtracted adsorption of poly-L-lysine can give the relative value of the interaction between tumor cells and DPPIV.

As shown in FIG. 4B and FIG. 4C, compared to control protein MBP, DP4A have better ability to inhibit the interaction between full-length DPPIV and LL2 or 4T1 tumor cells. This result indicates that DP4A can specifically inhibit the adhesion of suspended tumor cells to full-length DPPIV. Hence, metastasis of tumor cells to secondary organs via full-length DPPIV can be inhibited by DP4A.

### Example 5

### In Vivo Experimental Metastasis Assay with DP4A

4T1 tumor cells were treated with trypsin and then with FBS to inhibit the trypsinization reaction. Next, the treated cells were recovered in 20% FBS DMEM at 37° C. for 2 hr.

Next, 7.5 μg/ml of MBP-DP4A or control protein MBP were mixed with cells and reacted at 37° C. for 20 min. After cells were separated with a centrifuge and the supernatant was removed, the obtained cells (2×10⁵) were suspended in 200 μl of MBP-DP4A or control protein MBP to increase the concentration of MBP-DP4A or control protein MBP. Then, the obtained cell solution was concomitantly injected into BALB/C mice through tail vein to make the amount of MBP-DP4A or control protein MBP in blood being about 0.45 mg/kg. After 16 days, the mice were sacrificed for observing the lung thereof.

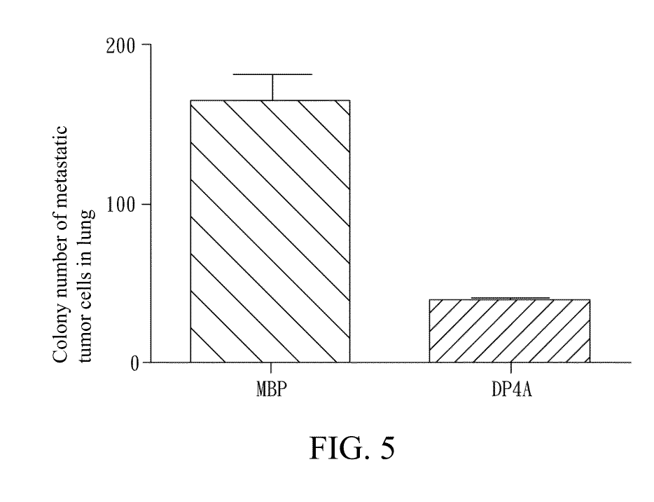

The tumor nodules in the lung of the mice were quantified with 3 mm/colony, and the obtained result is shown in FIG. 5. It is found that the metastatic tumor nodules in lung of MBP-DP4A group (DP4A) were one-fourth of that of MBP group (control protein MBP), and the weight of the lung of MBP-DP4A group was much lower than that of MBP group. Hence, DP4A can inhibit the metastasis of LL2 tumor cells to lung, so DP4A indeed have the ability to inhibit the tumor cell metastasis.

### Preparative Embodiment 2-1

### Preparation of Fluorescent Liposome

1.12 mg of dipalmitoylphosphatidylethanolamine (DPPE) was dissolved in a mixture of triethylamine (TEA) and chloroform (0.45 ml, TEA:chloroform=7:1000), and 1.48 mg of N-succinimidyl-S-acetylthioacetate was added therein (SATA) and stirred for 30 min. Next, 0.68 ml of chloroform was added in the solution, and the solution was dried with nitrogen. The aforesaid process was repeated twice. Finally, the resultant was dissolved with 0.5 ml of chloroform to form DPPE bonded with acetylthioacetate (ATA), i.e. DPPE-ATA.

Dipalmitoylphosphatidylcholine (DPPC, 13.42 mg), dipalmitoylphosphatidylglycerol (DPPG, 1.49 mg), cholesterol (6.96 mg) and lissamine rhodamine B-dipalmitoylphosphatidylethanolamine (LRB-DPPE, 150 μL (1 mg/mL) was placed in a flask and dissolved with chloroform/methanol (3/1, 1 ml). Then, DPPE-ATA (0.25 ml, 1.6 μmol) was added therein, and the organic solvent in the mixture was removed with nitrogen to obtain thin dry lipid film.

Sulforhodamine B (SRB, 0.2 mM) was dissolved in TBS buffer (20 mM Tris, 200 mM NaCl and pH 7.5), and pre-heated to 60° C. Next, the obtained thin dry lipid film was added therein to perform a hydrogenation, and the flask was placed in 60° C. water bath for 45 min. After sonication, the flask was placed in 60° C. water bath for 60 min.

A mini-extruder was pre-heated to 60° C., and the obtained solution was extruded polycarbonate film (pore size=50 nm) for 30 times. The obtained liposome was added onto a center of a surface of a resin column containing Sepharose CL4B resin. Sucrose buffer solution containing 25 mM Tris, 140 mM NaCl, and 51.77 g/L sucrose (pH 7.5) was used as elution buffer, and the eluents with violate color were collected in micro-centrifuge tubes. In addition, UV light may be used in the present experiment to determine the eluents where liposome containing SRB was contained therein. The obtained SRB-liposome was stored in dark and 4% TBS buffer containing 0.01% sodium azide.

### Preparative Embodiment 2-2

### Bonding of MBP to Liposome

The purified MBP (1 ml, 1 mg/ml, 23.81 nmol) was placed into a flask, and the pH thereof was kept in 7-9. Then, succinimidyl-[(N-maleimidopropionamido)-diethylene glycol]ester (SM(PEG)₂₄, 1.5 μl, 375.15 nmol) was added therein, and nitrogen was introduced therein. The solution was stirred at 160 rpm for 2 hr, and the unreacted SM(PEG)₂₄ (molecule weight=1.3 kDa) was separated by centrifugation with 1000 g to obtain SM (PEG)₂₄-tagged MBP.

The liposome prepared according to preparative embodiment 2-1 (6.15 mmol/ml, 0.97 ml diluted to 1 ml) was added into a flask, and 100 μl of hydroxylamine solution (0.5 M hydroxylamine hydrochloride, 25 mM EDTA, 0.1 M HEPES and pH 7.5) was added therein. Then, nitrogen was introduced therein for 1 min, and the solution was sonicated at room temperature for 2 hr (160 rpm) to perform deacetylation.

The deacetylated liposome was thiol-tagged liposome. The pH of the thiol-tagged liposome was adjusted to 6.5-7.5 with potassium phosphate, and then SM(PEG)₂₄-tagged MBP (pH 6.5-7.5) was added therein. After introducing nitrogen for 2 min, the solution was sonicated at room temperature of 3.5 hr, and then placed in a cold room overnight.

100 mM of N-ethylmaleimide solution was prepared with 0.02 M potassium phosphate, 0.15 M NaCl and sucrose buffer (pH 7.0). The obtained N-ethylmaleimide) solution (1.2 μl) was added into the overnight solution to make N-ethylmaleimide solution reacting with un-reacted thio-group. After sonication for 30 min, MBP-liposome encapsulated with SRB was separated with size exclusion chromatography, wherein the column is Sephadex CL-4B, and the balance buffer is TBS buffer containing sucrose.

### Preparative Embodiment 2-3

### Bonding of MBP-DP4 to Liposome

The process of the present embodiment is the same as that in Preparative Embodiment 2-2, except that the MBP was substituted with MBP-DP4A (1 ml, 0.5 mg/ml, 9 nmol), and the relative amount of SM(PEG)₂₄ (0.6 μl, 150 nmol) and liposome encapsulated with SRB (6.15 μmol/ml, 0.37 ml diluted to 1 ml) were adjusted.

### Example 6

### In Vivo Assay

Pericellular FN-positive cancer cells LL2 was used in the present in vitro assay to detect whether DP4A-liposome can enter into tumor cells via endocytosis thereof.

Adherent or suspended tumor cells were cultured in DMEM containing 20% FBS at 37° C. For 2 hr. The suspended tumor cells were firstly collected at room temperature with a centrifuge (1000 rpm, 3 min), and then the adherent or suspended tumor cells were washed with 1×PBS buffer. Next, tumor cells were treated with 0.5% BSA and MBP-liposome or DP4A-liposome with different concentration at different culture times.

The treated suspended tumor cells were firstly collected at room temperature by a centrifuge (1000 rpm, 3 min), and then the adherent or suspended tumor cells were washed with 1×PBS buffer for three times. Finally the tumor cells were fixed with 1% paraformaldehyde, and the endocytosis of tumor cells were visualized with Olympus fluorescent microscopy equipped with 1×2-ILL-100 Olympus Th4-100, HG-lamp (Olympus U-LH100HG) and CCD (Olympus DP71 and U-CMAD3).

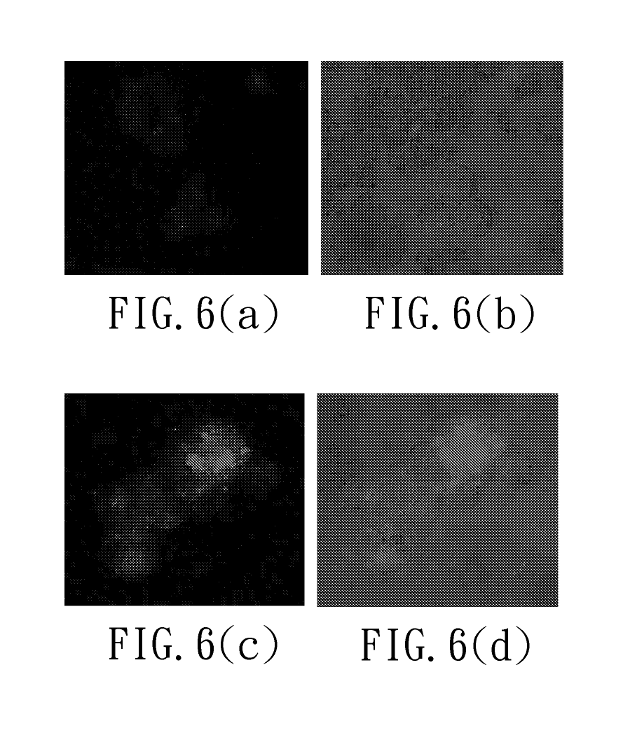

The results show that the best concentration and cultured time for DP4A-liposome entering into LL2 tumor cells via endocytosis is 0.7 μM and 2.5 hr. FIG. 6(a) is a fluorescent photo showing LL2 tumor cells treated with MBP-liposome; FIG. 6(b) is a fluorescent plus phase contrast photo showing LL2 tumor cells treated with MBP-liposome; FIG. 6(c) is a fluorescent photo showing LL2 tumor cells treated with DP4A-liposome; and FIG. 6(d) is a fluorescent plus phase contrast photo showing LL2 tumor cells treated with DP4A-liposome. As shown in FIG. 6(a) and FIG. 6(b), the liposome without DP4A cannot enter into LL2 tumor cells via endocytosis; but the liposome with DP4A can successfully enter into LL2 tumor cells via endocytosis, as shown in FIG. 6(c) and FIG. 6(d). These results indicate that the DP4A-liposome can be used to inhibit tumor cell growth or kill tumor cells when there are anti-cancer drugs contained in the DP4A-liposome.

### Example 7

### In Vivo Assay

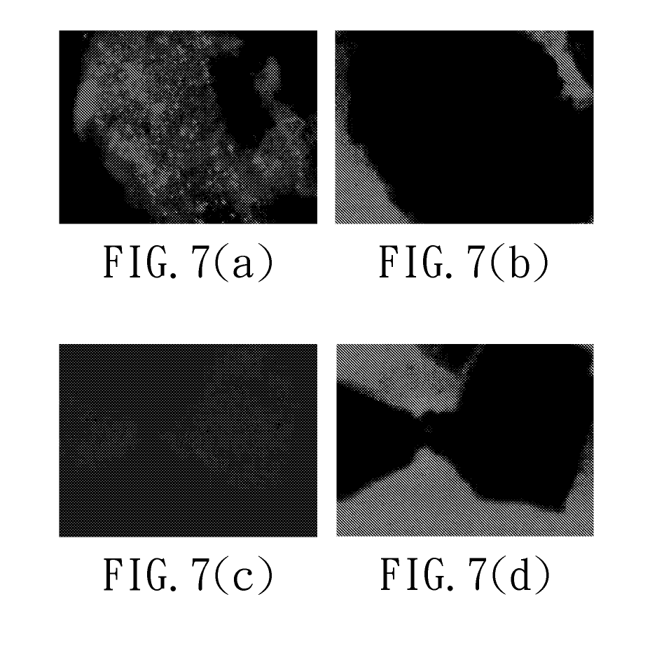

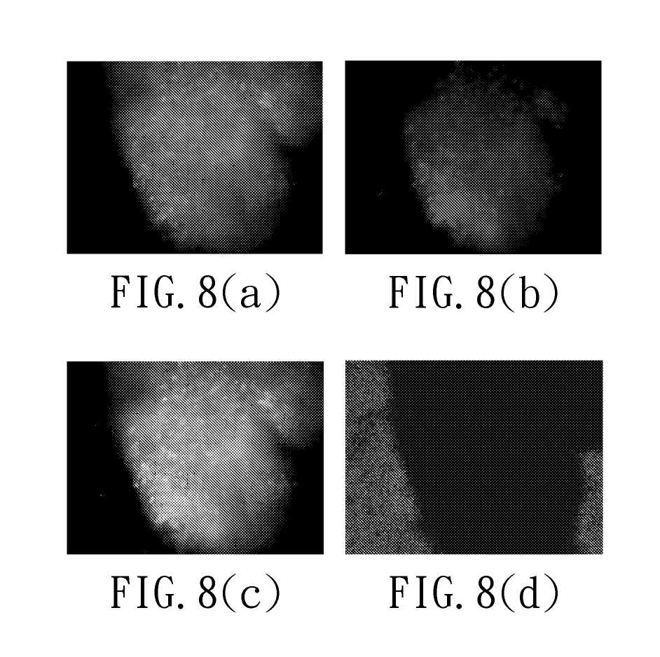

Metastatic tumor cells such as LL2 cells, which were or were not transfected with green fluorescent protein (GFP), were injected into mice through tail vein or fat pad. When the symptom of tumor metastasis became significant after 20 days, prepared MBP-liposome or DP4A-liposome (final volume as 200 μl, 0.7 μM) were injected into the mice through tail vein, After 2.5 hr, the mice were sacrificed, and the obtained organs were placed in 6-cm dishes. The organs were cut to form tumor tissue micro-pieces, and DMEM (500 μl) was added therein. Then, the states of tumor tissue micro-pieces absorbing liposome via endocytosis thereof were visualized with fluorescent microscopy. The results are shown in FIGS. 7(a)-7(d) and FIGS. 8(a)-8(d).

FIGS. 7(a)-7(d) show fluorescent and phase contrast photos of lung pieces when DP4A-liposomes or MBP-liposomes (final volume as 200 μl, 0.7 μM) were injected into the mice through tail vein after the metastasis of LL2 cells in mouse lungs was occurred. FIG. 7(a) is a fluorescent photo in which DP4A-liposome was intravenously injected after the metastasis of LL2 cells in the mouse lungs was occurred; FIG. 7(b) is a phase contrast photo in which DP4A-liposome was intravenously injected after the metastasis of LL2 cells in the mousse lungs was occurred; FIG. 7(c) is a fluorescent photo in which MBP-liposome was intravenously injected after the metastasis of LL2 cells in the mouse lungs was occurred; and FIG. 7(d) is a phase contrast photo in which MBP-liposome was intravenously injected after the metastasis of LL2 cells in the mouse lungs was occurred. As shown in FIGS. 7(a)-7(d), only DP4A-liposome can recognize metastasis tumor cells, and enter into tumor cells via endocytosis.

FIGS. 8(a)-8(d) show fluorescent and phase contrast photos of lung pieces when tumor cells transfected with GFP were injected into mice through tail vein and the metastasis of LL2 cells in the mouse lung was occurred. FIG. 8(a) is a fluorescent photo in which LL2 tumor cells were indicated by GFP; FIG. 8(b) is a fluorescence photo in which DP4A-liposomes were intracellularly delivered in the same LL2 cells as in FIG. 8(a) after metastasis of LL2 cells in the mouse lungs was occurred; FIG. 8(c) is a merged photo of tumor cells and DP4A-liposomes; and FIG. 8(d) is a phase contrast photo of lung pieces. As shown in FIGS. 8(a)-8(d), the yellow fluorescence is the merged part of DP4A-liposome and tumor cells, and the red fluorescence is not the self-fluorescence of tissues. This result indicates that the DP4A-liposome can recognize metastatic tumor cells.

The inventors observed not only the lung pieces, but also the pieces from marrow, liver, kidney, and lipid pad. The results show that the red fluorescence emitted from DP4A-liposome was observed in the primary tumor cells in lipid pad, and few colonies and little red fluorescence emitted from DP4A-liposome was also observed in other tissues of specific organs. These results indicate metastasis may also occur in these organs although this cannot be detected. Hence, DP4A-liposome of the present invention can be used to specifically treat not only primary cancers, but also other metastatic tumor cells including un-detected tumor cells to achieve the purpose of decreasing side effects. However, when DP4A-liposomes were injected into health mice without tumor cell injection, the fluorescence only can be observed in metabolically active organ such as liver and kidney, but cannot be observed in other organ pieces. In addition, there was no phenomenon of gathered fluorescence in organ pieces of health mice.

According to the above results, when the Sulforhodamine B encapsulated in the DP4A-liposome is substituted with a marker, the DP4A-liposome containing the marker can enter into tumor cells expressing fibronectin via endocytosis. Therefore, the tumor cells can be locally and specifically tagged and the tagged tumor cells can be used as a diagnosis reference for determining the condition or degree of metastasis.

### Preparative Embodiment 3

### Preparation of DP4A-Liposome or MBP-Liposome Containing Doxorubicin

The process for preparing DP4A-liposome or MBP-liposome containing doxorubicin was the same as those described in Preparative Embodiment 2-1 to 2-3, except that the Sulforhodamine B used in the Preparative embodiments 2-1 was substituted with doxorubicin (Dox) in the present preparative embodiment. The obtained MBP-liposome or DP4A-liposome encapsulating doxorubicin was shown in MBP-lipo-dox and DP4A-lipo-dox in the following example.

### Example 9

### In Vivo Assay

The process of the present example was the same as those described in Example 6, except that the MBP-liposome or DP4A-liposome encapsulated with SRB are substituted with the aforesaid MBP-lipo-dox and DP4A-lipo-dox. The concentration of MBP-lipo-dox or DP4A-lipo-dox used in the present example was 0.7 μM.

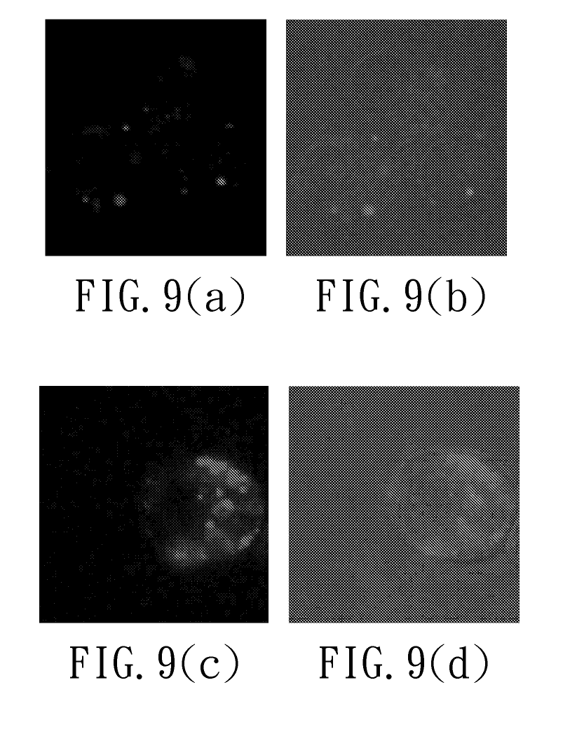

The results of the present example are shown in FIGS. 9(a)-9(d). FIG. 9(a) is a fluorescent photo showing LL2 tumor cells treated with DP4A-lipo-dox; FIG. 9(b) is a fluorescent plus phase contrast photo showing LL2 tumor cells treated with DP4A-lipo-dox; FIG. 9(c) is a fluorescent photo showing LL2 tumor cells treated with MBP-lipo-dox; and FIG. 9(d) is a fluorescent plus phase contrast photo showing LL2 tumor cells treated with DP4A-lipo-dox. As shown in FIG. 9(c) and FIG. 9(d), the liposome without DP4A cannot enter into LL2 tumor cells via endocytosis; but the liposome with DP4A can successfully enter into LL2 tumor cells via endocytosis, as shown in FIG. 9(a) and FIG. 9(b). These results indicate that the DP4A-lipo-dox can enter into tumor cells. In addition, the results also show that the endocytosis of DP4A-lipo-dox was dose-dependent (data not shown).

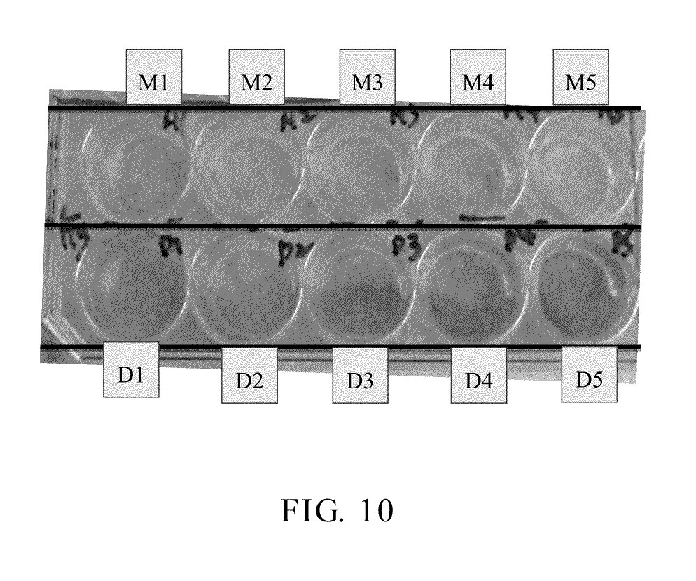

Furthermore, FIG. 10 is a photo of MTF7 tumor cells treated with MBP-lipo-dox and DP4A-lipo-dox respectively, wherein M1 to M5 dishes represented MTF7 tumor cells treated with 1/10 of fraction #1 to #5 from the chromatographic purification of MBP-lipo-dox, D1 to D5 dishes represented MTF7 tumor cells treated with 1/10 fraction 41 to #5 from the chromatographic purification of DP4A-lipo-dox, the approximate concentration of tractions #5 of both DP4A-lipo-dox and MBP-lipo-dox is 7 μM, and the concentrations of MBP-lipo-dox and DP4A-lipo-dox was apparently and respectively increased from M1 to M5 and D1 to D5. The yellower color means the better cell growth rate and the redder color means the worse cell growth rate. As shown in FIG. 10, as the concentration of DP4A-lipo-dox treating MTF7 tumor cells increased, more tumor cells died. This result indicates that the ability of DP4A-lipo-dox to kill tumor cells or inhibit cell growth is doe-dependent.

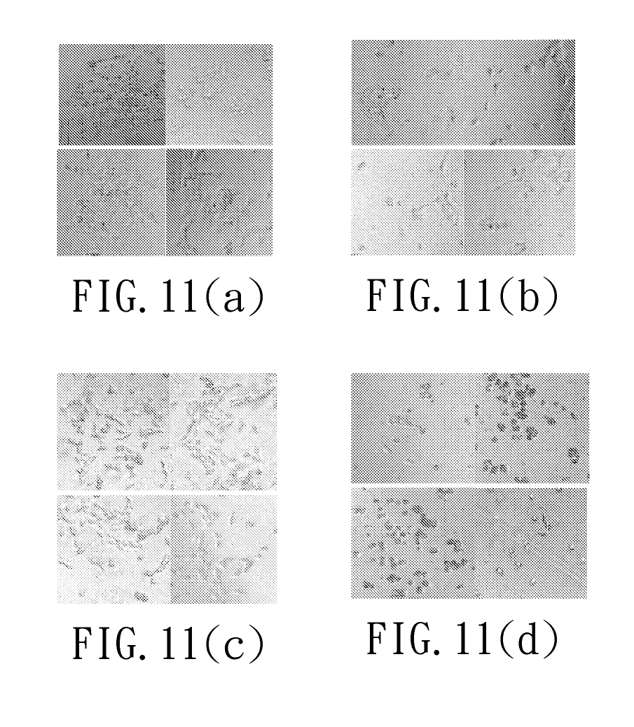

In addition, FIGS. 11(a)-11(b) show phase contrast photos of LL2 and 4T1 tumor cells treated with 0.7 μM MBP-lipo-dox or DP4A-lipo-dox, wherein FIG. 11(a) is a phase contrast photo of LL2 treated with MBP-lipo-dox, FIG. 11(b) is a phase contrast photo of LL2 treated with DP4A-lipo-dox, FIG. 11(c) is a phase contrast photo of 4T1 cells treated with MBP-lipo-dox, and FIG. 11(c) is a phase contrast photo of 4T1 cells treated with DP4A-lipo-dox. The results show that LL2 as well as 4T1 tumor cells treated with MBP-lipo-dox grew and spread properly, but those treated with DP4A-lipo-dox were rounded up showing signs of cell death. Hence, the DP4A-lipo-dox indeed has potential for cancer treatment.

### Example 10

### In Vivo Assay

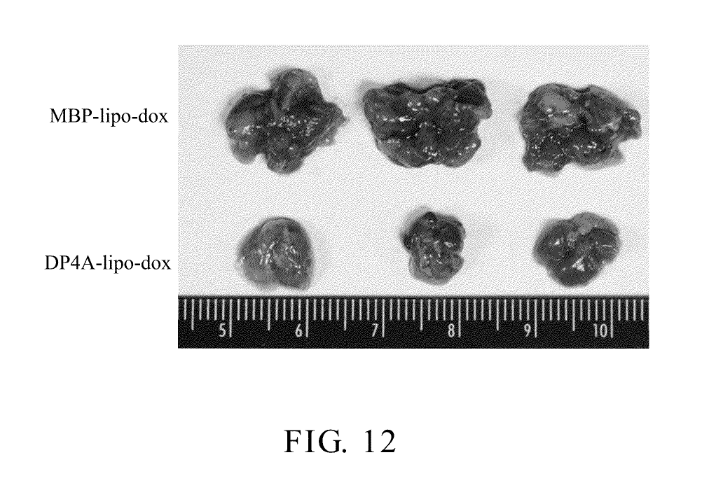

Metastatic tumor cells LL2 or 4T1 (5×105) in DMEM were injected into mice through tail vein. After 5 days, pre-prepared MBP-lipo-dox or DP4A-lipo-dox (final volume as 200 μl, 0.7 μM) were injected into the mice via intravenous tail vein injection. After another 7 days, pre-prepared MBP-lipo-dox or DP4A-lipo-dox (final volume as 200 μl, 0.7 μM) was injected into the mice via intravenous tail vein injection again. After the mice were sacrificed, the obtained lungs were placed in 6-cm dishes. The results are shown in FIG. 12. The results show that the tumor size in lungs of mice treated with DP4A-lipo-dox was much smaller than that treated with MBP-lipo-dox. Hence, the DP4A-lipo-dox of the present invention indeed can inhibit metastasis of tumor cells.

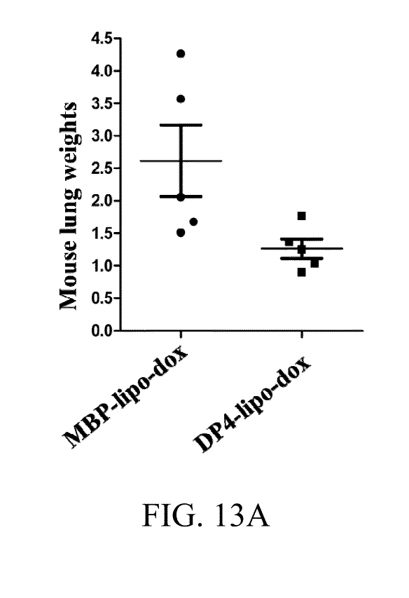

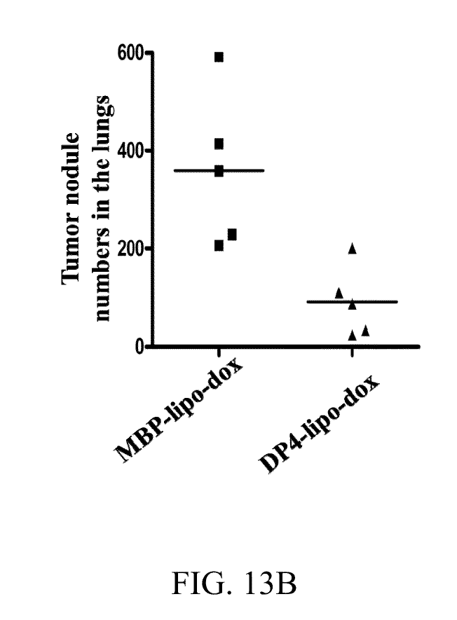

The quantitative results shown in FIG. 13A and FIG. 13B respectively show the mouse lung weights and the tumor nodule numbers in the lungs taken from mice with cancer metastases of the present example.

The results show that both the mouse lung weights and the tumor nodule numbers in the lungs taken from mice treated with DP4A-lipo-dox are much smaller than those treated with MBP-lipo-dox. Hence, DP4A/-lipo-dox used in the present example indeed has potential for inhibiting or treating metastasis.

In the aforesaid examples, doxorubicin is only one example of anti-cancer drug, but the present invention is not limited thereto.

According to the above results, when an active agent such as anti-cancer drug was encapsulated into DP4A-liposome of the present invention, the DP4A-liposome encapsulated active agent can enter into tumor cells expressing fibronectin via endosytosis. Therefore, the tumor cells can be locally and specifically treated with DP4A-liposome-active agent (i.e. the pharmaceutical composition of the present invention). Compared to the conventional chemotherapy for treating metastasis, which is non-specific and hurt normal cells easily, the pharmaceutical composition of the present invention can target metastatic tumor cells and obtain an effect of specifically treating.

Although the present invention has been explained in relation to its preferred embodiment, it is to be understood that many other possible modifications and variations can be made without departing form the spirit and scope of the invention as hereinafter claimed.

What is claimed is:

1. A method for reducing an established metastatic tumor, comprising administering an effective amount of a pharmaceutical composition to a subject in need, wherein the pharmaceutical composition comprises a polypeptide of a fibronectin-binding domain of dipeptidyl peptidase IV having a maltose-binding protein (MBP) fused at an N-terminal thereof, a cross-linking molecule, an active agent, and a pharmaceutically acceptable carrier, and wherein the cross-linking molecule is SM(PEG).sub.24, and the pharmaceutically acceptable carrier is liposome.

2. The method as claimed in claim 1, wherein the fibronectin-binding domain of dipeptidyl peptidase IV has a sequence with 70-100% similarity to a sequence represented by SEQ ID NO: 1.

3. The method as claimed in claim 1, wherein the fibronectin-binding domain of dipeptidyl peptidase IV has a sequence with 70-100% identity to a sequence represented by SEQ ID NO: 1.

4. The method as claimed in claim 1, wherein the fibronectin-binding domain of dipeptidyl peptidase IV has a sequence represented by SEQ ID NO: 1.

5. The method as claimed in claim 1, wherein the fibronectin-binding domain of dipeptidyl peptidase IV is a binding domain A of dipeptidyl peptidase IV.

6. The method as claimed in claim 1, wherein the active agent is doxorubicin.

7. The method as claimed in claim 1, wherein the liposome is ATA-tagged liposome.


SEQUENCE LISTING (3 sequences)

SEQ ID NO: 1 - protein, 102 aa, Rattus norvegicus
  ref <308>: DATABASE ACCESSION NUMBER: NCBI/NP_036921.1
  ref <309>: DATABASE ENTRY DATE: 2011-04-10
  ref <313>: RELEVANT RESIDUES IN SEQ ID NO: (29)..(130)
NKDEAAADSRRTYTLADYLKNTFRVKSYSLRWVSDSEYLYKQENNILLFNAEHGNSSIFLENSTFEIFGDSISDYSVSPDRLFVLLEYNYVKQWRHSYTASY

SEQ ID NO: 2 - DNA, 306 bp, Rattus norvegicus
  ref <308>: DATABASE ACCESSION NUMBER: NCBI/NP_036921.1
  ref <309>: DATABASE ENTRY DATE: 2011-04-10
  ref <313>: RELEVANT RESIDUES IN SEQ ID NO: (85)..(390)
aacaaagatgaagcggccgctgatagccgcagaacttacacactagctgactatttaaagaatacctttcgggtcaagtcctactccttgcggtgggtttcagattctgaatacctctacaagcaagaaaacaatatcttgctattcaatgctgaacacgggaacagctccattttcttggagaacagtacctttgagatctttggagattctataagtgattattcagtgtcaccggacagactgttcgttctcttagaatacaattatgtgaagcaatggagacactcctacacggcttcatac

SEQ ID NO: 3 - protein, 495 aa, unknown
  MBP-DP4A
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFNKDEAAADSRRTYTLADYLKNTFRVKSYSLRWVSDSEYLYKQENNILLFNAEHGNSSIFLENSTFEIFGDSISDYSVSPDRLFVLLEYNYVKQWRHSYTASYPR